(19) United States
(12) Patent Application Publication
(10) Pub. No.: US 2009/0231544 A1
(43) Pub. Date: Sep. 17, 2009

(54) Affordable eyeglass fitting method and system

(76) Inventor: Gregory Mahloch, Omaha, NE (US)

Correspondence Address: STINSON MORRISON HECKER LLP;ATTN: PATENT GROUP, 1201 WALNUT STREET, SUITE 2800, KANSAS CITY, MO, 64106-2150, US

(21) Appl. No.: 12/077,006

(22) Filed: Mar. 14, 2008

(51) Int. Cl. G02C 13/00; A61B 3/036

(52) U.S. Cl. 351/246; 351/178

(57) ABSTRACT

A method for providing eyeglasses for persons of limited means which includes the steps of providing an eyeglass frame including left and right circular lens support rims, a width-adjustable bridge and a pair of adjustable temples and a plurality of plastic preformed circular lenses, each fitting into the lens support rims and having two lens components, a spherical lens component and a cylindrical lens component. A left and a right preformed circular lens are selected from the plurality of preformed circular lenses which approximate the preferred spherical and cylindrical lens corrective strengths, and these are inserted and rotated in the rims, then it is determined whether the selected lenses provide adequate vision correction. These steps are repeated until the selected left and right preformed circular lenses provide adequate vision correction to the person being fitted.

### CROSS-REFERENCE TO RELATED APPLICATIONS

Not Applicable.

### STATEMENT REGARDING FEDERALLY SPONSORED RESEARCH OR DEVELOPMENT

Not applicable.

### BACKGROUND OF THE INVENTION

1. Field of the Invention

The present invention is directed to eyeglass fitting and providing methods and, more particularly, to a method for providing eyeglasses for persons of limited means which includes the steps of providing at least one (1) plastic eyeglass frame having a frame front including left and right circular lens support rims, a width-adjustable bridge and a pair of adjustable temples, further providing a plurality of plastic preformed circular lenses, each having a center axis and a diameter approximating the inner diameter of the two (2) circular lens support rims to be secured therein, each of the circular lenses having two (2) lens components, a spherical lens component having a predetermined strength and a cylindrical lens component having a predetermined strength and orientation, the plurality of preformed circular lenses including lenses having different strengths of spherical and cylindrical lens components, approximating the preferred spherical lens corrective strength and preferred cylindrical lens corrective strength and orientation, selecting a left and a right preformed circular lens from the plurality of preformed circular lenses which approximate the preferred spherical and cylindrical lens corrective strengths, inserting the selected left and right preformed circular lenses into the lens support rims of the plastic eyeglass frame, rotating the left and right preformed circular lenses about their center axes within the support frames to generally align the cylindrical lens component for correction of the astigmatism for the eye, placing the eyeglass frame including the selected left and right circular lenses onto the head of the person being fitted, adjusting the width adjustable bridge and adjustable temples to generally align the lenses with the eyes of the person whose vision is being corrected, determining if the selected left and right preformed circular lenses provide adequate vision correction and repeating the selecting, inserting, rotating, placing, adjusting and determining steps until the selected left and right preformed circular lenses provide adequate vision correction to the person being fitted with eyeglasses.

2. Description of Related Art

When a person is being fitted for new eyeglasses or replacement eyeglasses, he or she generally undertakes similar steps regardless of the optometrist's office which he or she visits. In general, the person being fitted for the lenses would visit the optometrist or ophthalm ologist who would sit them in a darkened room and have the patient look through a device called a phoropter, which includes many different strengths and configurations of lenses which are used to determine the best fit for corrective lenses to be used for the person being fitted. The ophthalmologist or optometrist would then take lens blanks and insert them into an ophthalmic lens grinding system which quickly and accurately grinds the lens blank to the size, shape and configuration desired by the ophthalmologist, optometrist and/or person being fitted. The resulting lenses are then inserted into a selected pair of frames, secured therein and the frames are then placed on the person's face for final adjustment of the nose pieces and/or the frame temples. The completed eyeglasses are then ready to be worn by the person being fitted therefor.

However, while these fitting methods are perfectly acceptable for persons who have access to such facilities, it is virtually impossible for persons of limited financial means or who are living in third world countries to obtain this same access to fitting methods, and therefore it becomes a far more difficult matter to obtain affordable, properly fitted eyeglasses for such individuals. There is, therefore, a need for an eyeglass fitting and providing method which may be provided to persons on limited means or persons who do not have access to ophthalmologists or optometrists.

There is, therefore, a need for a method for providing eyeglasses for persons of limited means which addresses and solves the deficiencies currently found in eyeglass fitting methods and systems.

Another object of the present invention is to provide a method for providing eyeglasses for persons of limited means which includes the steps of providing at least one (1) eyeglass frame, preferably constructed of plastic, having a frame front including left and right circular lens support rims, a width adjustable bridge and a pair of adjustable temples, the plastic eyeglass frame being inexpensively constructed in order to keep the cost of use as low as possible thus making access to the present invention affordable to a larger group of underprivileged persons.

Another object of the present invention is to provide a method for providing eyeglasses for persons of limited means which includes the step of providing a plurality of plastic preformed circular lenses, each having a center axis and a diameter to fit and be secured within the lens support rims, each of the plurality of circular lenses having two (2) lens components, a spherical lens component having a predetermined strength and a cylindrical lens component having a predetermined strength and orientation, the plurality of preformed circular lenses including lenses having different strengths of spherical and cylindrical lens components, each of the lenses further including insignia formed on the lens to delineate the orientation of the cylindrical lens component.

Another object of the present invention is to provide a method for providing eyeglasses for persons of limited means which includes the step of approximating via visual acuity examination of the person to be fitted for eyeglasses the preferred spherical and cylindrical lens corrective strength and orientation to provide corrected vision for the person and selecting a preformed left and right circular lens from the plurality of preformed circular lenses which approximates the preferred spherical and cylindrical lens corrective strength for the left and right eyes of the person being fitted for eyeglasses.

Another object of the present invention is to provide a method for providing eyeglasses for persons of limited means which involves the steps of inserting the selected left and right preformed circular lenses into the circular lens support rims, rotating the left and right preformed circular lenses about the center axis thereof to generally align the cylindrical lens component for correction of the astigmatism for the eye, placing the eyeglass frame on the head of the person being fitted, adjusting the width adjustable bridge and adjustable temples to fit the lenses to the eyes, determining if adequate vision correction is provided by the fitted lenses and repeating the aforementioned steps until the selected left and right preformed circular lenses provide adequate vision correction to the person being fitted with the eyeglasses.

Another object of the present invention is to provide an improved method for fitting eyeglasses for persons of limited means which can be used in locations other than an ophthalmologist or optometrist office, thus permitting persons unable to visit an ophthalmologist or optometrist to be fitted for eyeglasses.

Another object of the present invention is to provide a method for fitting eyeglasses for persons of limited means which provides inexpensive fitted eyeglasses for such persons thus allowing them to read, see more clearly and live better and safer lives.

Finally, an object of the present invention is to provide a method for fitting eyeglasses for persons of limited means which is relatively simple and straightforward in use and provides safe and effective fitted eyeglasses for such persons.

### BRIEF SUMMARY OF THE INVENTION

The present invention provides a method for providing eyeglasses for persons of limited means which includes the steps of providing at least one eyeglass frame, preferably constructed of plastic, having a frame front including left and right circular lens support rims and a width-adjustable bridge extending between and connecting the left and right circular lens supports and a pair of adjustable temples mounted on opposite sides of the frame front and extending rearwards therefrom to extend over the ears of the wearer and providing a plurality of plastic preformed circular lenses each having a center axis and a diameter to fit and be secured within the two circular lens support rims, each of the plurality of circular lenses having two lens components, the first being a spherical lens component having a predetermined strength and the second being a cylindrical lens component having a predetermined strength and orientation, the plurality of preformed circular lenses including lenses having different strengths of spherical and cylindrical lens components. The next step is to approximate via visual acuity examination the preferred spherical lens corrective strength of the person to be fitted for eyeglasses to correct the refractive error of the eye and the preferred cylindrical lens corrective strength and orientation to correct the astigmatism of the eye to provide corrected vision for the person. Left and right preformed circular lenses would then be selected from the plurality of preformed circular lenses, the left and right preformed circular lenses having spherical and cylindrical lens strengths approximating the preferred spherical and cylindrical lens corrective strength for the left and right eyes of the person being fitted for eyeglasses. The selected left and right preformed circular lenses would then be inserted into the two circular lens support rims of the at least one plastic eyeglass frame, and the selected left and right preformed circular lenses are then rotated about the center axis within the two circular lens support frames to generally align the cylindrical lens alignment component for correction of the astigmatism for the eye. The plastic eyeglass frame including the selected left and right preformed circular lenses is then placed onto the head of the person being fitted with the eyeglasses and the width-adjustable bridge and the adjustable temples are adjusted to generally align the at least one plastic eyeglass frame on the head of the person being fitted such that selected left and right preformed circular lenses are generally aligned with the eyes for which vision is to be corrected. It is then determined if the selected left and right preformed circular lenses provide adequate vision correction to the person being fitted with the eyeglasses, and if not, the selecting, inserting, rotating, placing, adjusting and determining steps are repeated until the selected left and right preformed circular lenses provide adequate vision correction to the person being fitted with the eyeglasses.

The present invention as thus described provides many improvements to the fitting systems and methods currently found in the eyeglass fitting industry. For example, because round lenses and round frames are provided, it is a relatively simple manner to manufacture frames and lenses which quickly and accurately fit together to facilitate the fitting process. Furthermore, because the method of the present invention may be used in areas which are not generally visited by ophthalmologists and/or optometrists, a far wider range of humanity may be serviced by use of the present invention, thus fulfilling the desires and needs of underprivileged persons to obtain a set of eyeglasses to improve their quality of life. Finally, because the method of the present invention provides extremely low cost fitted eyeglasses to persons of limited means, a far greater percentage of society may receive corrective lenses and frames than is currently available on the market. It is thus seen that the method for fitting eyeglasses of the present invention provides significant improvements over those methods and systems found in the prior art.

### DESCRIPTION OF THE DRAWINGS

FIGS. 1 and 2 are perspective views of the eyeglass frames and lens case, respectively, of the present invention;

FIG. 3 is a detail side elevational view of the eyeglass frame with the lens being inserted therein;

FIG. 4 is a perspective view of the lens fitting system of the present invention being applied to a person being fitted for eyeglasses;

FIG. 5 is a front elevational view of the frames of the present invention showing the elements thereof.

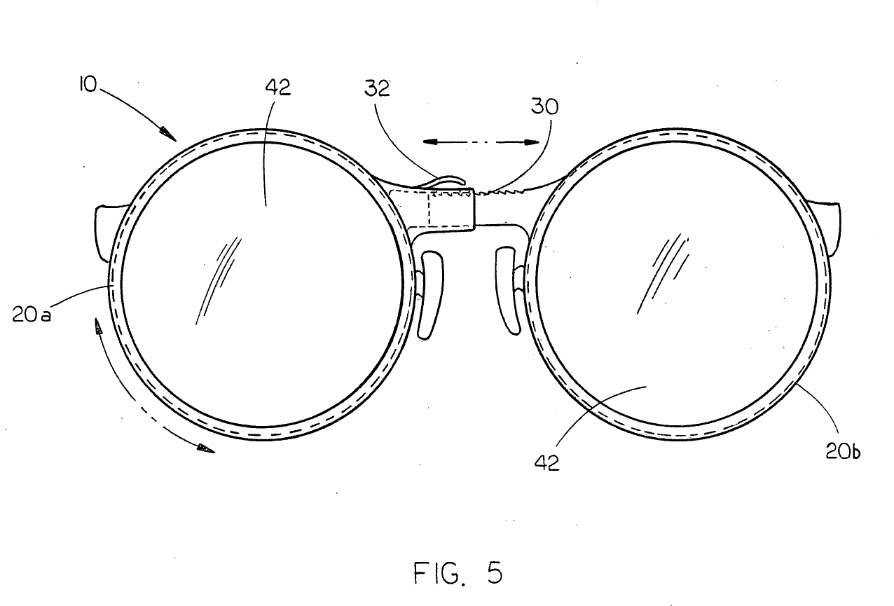

### DETAILED DESCRIPTION OF PREFERRED EMBODIMENT

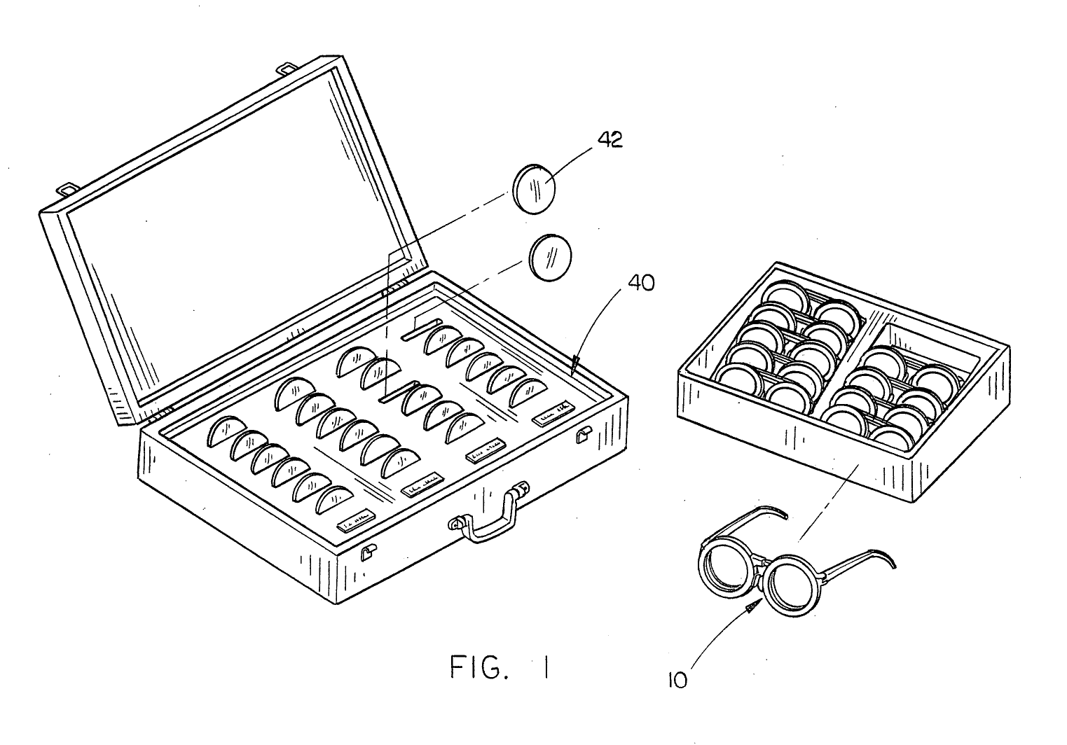

The method for providing eyeglasses of the present invention is shown best in FIGS. 1-4 as including the following steps. Initially, it should be noted that the present invention is designed to be used in situations where standard eyeglass fitting methods are either unavailable or extremely difficult to implement, such as would be encountered by aid workers or other such persons actually visiting locations where underprivileged persons are living. It is expected that the initial steps of the present invention of providing at least one (1) plastic eyeglass frame 10 having a frame front including left and right circular lens support rims 20a and 20b, a width-adjustable bridge 30 including an adjustment mechanism 32 and a pair of adjustable temples 34a and 34b mounted on opposite sides of the frame front would be accomplished by providing an injection-molded plastic eyeglass frame having a bridge which may be adjusted inwards and outwards to increase or decrease the distance between the left and right circular lens support rims 20a and 20b, and the pair of adjustable temples 34a and 34b may either be length-adjustable or may be manufactured of a semi-flexible plastic material which may be bent to generally conform to the ear on which the adjustable temple is to be supported. It should be noted that the frames 10 may be constructed of other materials than plastic, such as metal or other such materials, but it has been found that plastic frames are inexpensive to produce and also have a generally long lifespan, and therefore are preferred for use with the present invention.

In a preferred embodiment as shown in FIG. 2, the adjustable bridge 30 would preferably be a sliding connection between the two halves of the frame with a frictional securement device such as a pressure clip 32 releasably securing the bridge 30 in a selected position in order to permit adjustment of the distance between the rims and thus provide for adjustment of the width of the glasses to coordinate with the distance between each individuals pupils, commonly referred to as pupillary distance.

The pupillary distance is the distance between the centers of the pupils in each eye, and this measurement is used when making prescription eyeglasses to properly position the lenses correctly in relation to the pupil centers, as is particularly critical in connection with higher powered lenses. It has been found that construction of the plastic frames 10 including the adjustable bridge 30 will not significantly increase the construction cost for the frames yet will provide a critical adjustment feature which will allow the same frame design to be used in connection with many different individuals being fitted for glasses.

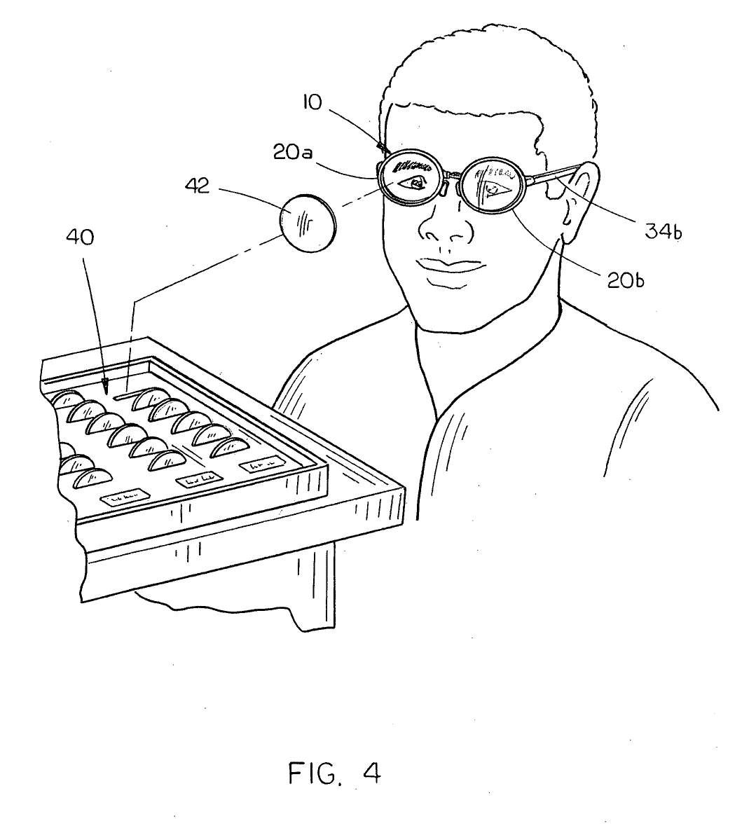

Once the standard pair of eyeglass frames 10 has been assembled, the fitting method of the present invention may be implemented to quickly and easily provide low-cost eyeglasses for persons of limited means and/or who cannot easily visit an ophthalmologist or optometrist. Specifically, the person being fitted for eyeglasses, as shown in FIG. 4, would initially undergo a field examination to generally determine his or her vision characteristics, and such field vision tests are well-known in the field of optometry. In the most common type of test, the individual being fitted for glasses would read from an eye chart to determine their general level of nearsightedness or farsightedness and then through the use of a spoke diagram or the like, the direction and level of astigmatism in the person being fitted for eyeglasses may be generally determined. Once the general vision parameters of the person being fitted for eyeglasses have been determined via the field tests, the lens case 40 is opened to select the appropriate lenses for the left and right eyes based on the vision testing previously done.

As shown best in FIG. 1, the lens case 40 would preferably be a suitcase-type enclosure case which includes a plurality of plastic preformed circular lenses 42 each mounted within the lens case 40 in a series of lens holders 44 which support the lenses 42 to prevent breaking and/or scratching of the lenses 42 when held within lens case 40. In a preferred embodiment, the lens case 40 would be subdivided into a plurality of compartments in which lenses 42 having similar configurations would be mounted. For example, the majority of people to be fitted with eyeglasses fall within a certain range of lens strength and astigmatism correction. Specifically, the average range for correction is −1.00 to −4.00 with the average correction being approximately −2.00. Therefore, it would be advantageous to supply a plurality of lenses which vary between −1.00 to −4.00 in increments of 0.25 strength, with the median part of the range of strengths including additional lenses as it is expected that more persons being fitted for lenses will required corrections within the average range than outside the average range. Furthermore, although the above description is intended for spherical lens correction values, the same steps would be applied to the cylindrical lens correction values for astigmatism, namely that the majority of persons being fitted for glasses have a small degree of astigmatism which needs to be corrected and, therefore, the lenses provided within lens case 40 would include more lenses having small degrees of correction for astigmatism than lenses including a large degree of correction for astigmatism. This allows the user of the present invention to fit the glasses to a majority of persons, as it is highly likely that the majority of people being fitted with the glasses would fall within the ranges of lenses 42 held within lens case 40. Of course, the benefit of the present invention is that other lenses having higher correction values may be included in the lens case 40, it being less likely, however, that those lenses will be used during the fitting process. Should the need for use of those arise, however, they will be available within lens case 40, and thus persons requiring significant vision correction may be served by the method of the present invention as well as those falling more within the average range of correction as was discussed previously.

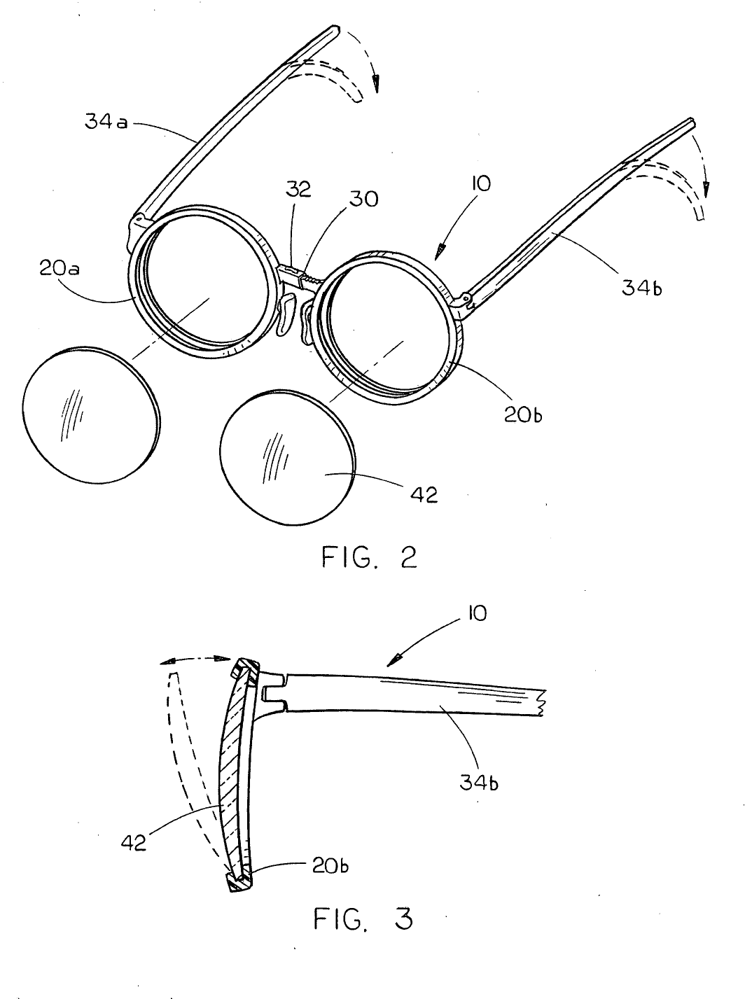

Once the left and right preformed circular lenses 50a and 50b have been selected from the lens case 40, they are “popped” or fitted into the eyeglass frame 10 which deforms slightly to receive and retain the lenses 50a and 50b therein due to the semi-flexible nature of the plastic material from which the eyeglass frame 10 is manufactured, as shown in FIG. 3. Furthermore, because the plastic material has a reasonable high coefficient of friction, the lenses 50a and 50b are retained within the eyeglass frame 10 once they are placed therein, particularly through frictional contact between the circular lens support rims 20a and 20b and the lenses 50a and 50b, until such time as the lenses 50a and 50b need to be removed therefrom.

The left and right preformed circular lenses 50a and 50b would then be rotated within the circular rims 20a and 20b to initially align the cylindrical lens component for correction of the field test-determined astigmatism for the individual being fitted for the glasses. To this end, each of the left and right preformed circular lenses 50a and 50b, and in fact all of the lenses 42 used in connection with the present invention, would include a marker or insignia which delineates the alignment of the cylindrical lens component within the lens itself. This marking facilitates rotation of the lenses 50a and 50b to a preferred alignment of the cylindrical lens component of the preformed circular lens in order to provide astigmatism correction for the individual being fitted for the glasses.

The eyeglass frame 10 would then be placed on the head of the person being fitted with the eyeglasses and the bridge 30 would be adjusted via adjustment device 32 to generally align the circular lenses 50a and 50b with the pupil centers, thus aligning the distance between the lenses 50a and 50b with the pupillary distance of the individual being fitted. For informational purposes, the pupillary distance will generally fall between 58 mm and 65 mm, with the average being 62 mm, although larger and smaller distances between the pupils have been found. Therefore, it is seen that the adjustment device 32 on bridge 30 need not provide a significant distance of adjustment, but rather merely permit fine adjustment of the distance between the circular rims 20a and 20b and thus lenses 50a and 50b to properly align the lenses 50a and 50b with the pupils of the individual being fitted. Likewise, the temples 34a and 34b would be adjusted to better fit on the individual being fitted, with the adjustment being likely performed by bending of the temples 34a and 34b to conform to the ears of the individual being fitted.

The person being fitted for eyeglasses would then be asked to again read from an eye chart and undergo astigmatism testing to determine if the selected left and right preformed circular lenses provide adequate vision correction. If it is determined that the selected lenses 50a and 50b do provide adequate vision correction, the fitting process is completed and the individual thus has a new inexpensive pair of eyeglasses ready for use immediately. On the other hand, should it be determined that the selected lenses 50a and 50b do not provide adequate vision correction, the above-described steps would merely be repeated to obtain the correct lenses and lens orientations to provide the best vision correction available via use of the present invention. Regardless of the number of iterations required to obtain the correct preformed circular lenses 50a and 50b for use with the eyeglass frames 10, it is very important to note that each and every one of these steps is being performed without requiring a visit to an ophthalmologist or optometrist, and furthermore, the series of steps which must be undertaken to fit a person with eyeglasses using the present invention may be quickly and easily learned by persons not necessarily trained in the field of optometry. Therefore, individuals such as aid workers, peace corps participants or members of the armed services may be quickly and easily trained in the use of the method of the present invention to provide eyeglasses to persons in locations and countries which would not otherwise be able to either obtain or afford eyeglasses. It is thus clear that the present invention provides numerous advantages over the prior methods and systems for providing eyeglasses.

It is to be understood that numerous additions, modifications and substitutions may be made to the affordable eyeglass fitting method and system of the present invention which fall within the intended broad scope of the appended claims. For example, the precise size, shape and construction materials used in connection with eyeglass frame 10 and lenses 42 may be modified or changed so long as the cost is kept as low as is practicable and the circular orientation of the lenses and rims is generally maintained to provide a standard configuration for the lenses and rims. Furthermore, because the present invention may be used by persons not specifically trained as optometrists, far more persons may be served by use of the present invention than presently available in connection with fitting methods and systems found in the prior art, and, therefore, a much broader range of humanity may be served by the use of the present invention than that available previously. Finally, because the method of the present invention provides low cost eyeglasses for persons not otherwise able to obtain eyeglasses, the present invention provides significant benefits to those persons and certainly will improve their quality of life, which renders the present invention an important and beneficial method for fitting and providing eyeglasses.

There has therefore been shown and described an affordable eyeglass fitting method and system which accomplishes at least all of its intended objectives.

I claim:

1. A method for providing eyeglasses for persons of limited means comprising the steps:
providing at least one eyeglass frame having a frame front including left and right circular lens support rims and a width-adjustable bridge extending between and connecting said left and right circular lens supports and a pair of adjustable temples mounted on opposite sides of said frame front and extending rearwards therefrom to extend over the ears of the wearer;
providing a plurality of plastic preformed circular lenses each having a center axis and a diameter to fit and be secured within said two circular lens support rims, each of said plurality of circular lenses having two lens components, a spherical lens component having a predetermined strength and a cylindrical lens component having a predetermined strength and orientation, said plurality of preformed circular lenses including lenses having different strengths of spherical and cylindrical lens components;
approximating via visual acuity examination of a person to be fitted for eyeglasses the preferred spherical lens corrective strength to correct the refractive error of the eye and the preferred cylindrical lens corrective strength and orientation to correct the astigmatism of the eye to provide corrected vision for the person;
selecting a left and right preformed circular lens from said plurality of preformed circular lenses, said left and right preformed circular lens having spherical and cylindrical lens strengths approximating the preferred spherical and cylindrical lens corrective strength for the left and right eyes of the person being fitted for eyeglasses;
inserting said selected left and right preformed circular lenses into said two circular lens support rims of said at least one eyeglass frame;
rotating said selected left and right preformed circular lenses about said center axis within said two circular lens support frames to generally align said cylindrical lens alignment component for correction of the astigmatism for the eye;
placing said at least one eyeglass frame including said selected left and right preformed circular lenses onto the head of the person being fitted with said eyeglasses;
adjusting said width-adjustable bridge and said adjustable temples to generally align said at least one eyeglass frame on the head of the person being fitted such that selected left and right preformed circular lenses are generally aligned with the eyes for which vision is to be corrected;
determining if the selected left and right preformed circular lenses provide adequate vision correction to the person being fitted with said eyeglasses; and
repeating said selecting, inserting, rotating, placing, adjusting and determining steps until said selected left and right preformed circular lenses provide adequate vision correction to the person being fitted with said eyeglasses.

2. The method of claim 1 wherein each of said plurality of plastic preformed circular lenses further comprises an insignia imprinted thereon which delineates the alignment of the cylindrical lens component within each of said plurality of plastic preformed circular lenses thereby facilitating rotation of said lenses to a preferred alignment of the cylindrical lens component of the preformed circular lens in order to provide astigmatism correction for the individual being fitted for the glasses.

3. The method of claim 1 wherein said at least one eyeglass frame is constructed of plastic.

4. The method of claim wherein said step of determining if the selected left and right preformed circular lenses provide adequate vision correction to the person being fitted with said eyeglasses comprises having the person being fitted read from an eye chart to determine their corrected level of nearsightedness or farsightedness and then view a spoke diagram to relate the direction and level of corrected astigmatism in the person being fitted for eyeglasses.

5. A method for providing eyeglasses for persons of limited means comprising the steps:
providing at least one plastic eyeglass frame having a frame front including left and right circular lens support rims and a width-adjustable bridge extending between and connecting said left and right circular lens supports and a pair of adjustable temples mounted on opposite sides of said frame front and extending rearwards therefrom to extend over the ears of the wearer;
providing a plurality of plastic preformed circular lenses each having a center axis and a diameter to fit and be secured within said two circular lens support rims, each of said plurality of circular lenses having two lens components, a spherical lens component having a predetermined strength and a cylindrical lens component having a predetermined strength and orientation, said plurality of preformed circular lenses including lenses having different strengths of spherical and cylindrical lens components, each of said plurality of plastic preformed circular lenses further including an insignia imprinted thereon which delineates the alignment of the cylindrical lens component within each of said plurality of plastic preformed circular lenses thereby facilitating rotation of said lenses to a preferred alignment of the cylindrical lens component of the preformed circular lens in order to provide astigmatism correction for the individual being fitted for the glasses;
approximating via visual acuity examination of a person to be fitted for eyeglasses the preferred spherical lens corrective strength to correct the refractive error of the eye and the preferred cylindrical lens corrective strength and orientation to correct the astigmatism of the eye to provide corrected vision for the person;
selecting a left and right preformed circular lens from said plurality of preformed circular lenses, said left and right preformed circular lens having spherical and cylindrical lens strengths approximating the preferred spherical and cylindrical lens corrective strength for the left and right eyes of the person being fitted for eyeglasses;
inserting said selected left and right preformed circular lenses into said two circular lens support rims of said at least one plastic eyeglass frame;
rotating said selected left and right preformed circular lenses about said center axis within said two circular lens support frames to generally align said cylindrical lens alignment component for correction of the astigmatism for the eye;
placing said at least one plastic eyeglass frame including said selected left and right preformed circular lenses onto the head of the person being fitted with said eyeglasses;
adjusting said width-adjustable bridge and said adjustable temples to generally align said at least one plastic eyeglass frame on the head of the person being fitted such that selected left and right preformed circular lenses are generally aligned with the eyes for which vision is to be corrected;
determining if the selected left and right preformed circular lenses provide adequate vision correction to the person being fitted with said eyeglasses; and
repeating said selecting, inserting, rotating, placing, adjusting and determining steps until said selected left and right preformed circular lenses provide adequate vision correction to the person being fitted with said eyeglasses.

6. The method of claim 5 wherein said at least one eyeglass frame is constructed of plastic.

7. The method of claim 5 wherein said step of determining if the selected left and right preformed circular lenses provide adequate vision correction to the person being fitted with said eyeglasses comprises having the person being fitted read from an eye chart to determine their corrected level of nearsightedness or farsightedness and then view a spoke diagram to relate the direction and level of corrected astigmatism in the person being fitted for eyeglasses.
